(19) United States
(12) Patent Application Publication
(10) Pub. No.: US 2025/0339969 A1
(43) Pub. Date: Nov. 6, 2025

(54) ROBOTIC CALIBRATION

(71) Applicant: Brainlab AG, Munich (DE)

(72) Inventor: Stephan NOWATSCHIN, Munich (DE)

(21) Appl. No.: 18/855,367

(22) PCT Filed: May 24, 2022
(86) PCT No.: PCT/EP2022/063998

§ 371 Date: Oct. 9, 2024

(51) Int. Cl. B25J 9/16; A61B 34/32; A61B 34/37

(52) U.S. Cl. CPC B25J 9/1692; A61B 34/32; A61B 34/37; B25J 9/1697

(57) ABSTRACT

The present disclosure relates to an approach for calibrating a motorized medical support structure such as a medical robotic arm with respect to medical appliances and devices, wherein the support structure acts automatically so as to reach a calibration feature of a medical device it is to be calibrated with.

### FIELD OF THE INVENTION

The present invention relates to a computer-implemented method of automatically calibrating a motorized medical support structure, a corresponding computer program, a computer-readable storage medium storing such a program and a computer executing the program, as well as a medical system comprising the aforementioned computer.

### TECHNICAL BACKGROUND

In the field of medical technology, robotic systems have been found to provide valuable assistance as they are capable of performing tasks which are considered too demanding for humans, such as holding and guiding medical instruments over a long period of time and with absolute precision. Before the start of a medical procedure with robot assistance, it needs to be ensured that the robotic system along with its coordinate system it is working with is calibrated and registered with respect to the remaining technical setup for the procedure, such that the cooperation of all medical appliances and devices is free from any spatial miscalculations which may have serious effect on the outcome of the procedure.

A common approach of calibrating instruments and devices is to bring predefined sections of such instruments and devices into alignment with a reference structure or a calibration feature having a known spatial position (spatial location and/or spatial orientation). With the predefined section resting in the known spatial position, its position relative to other structures such as tracking markers can initially be defined and tracked afterwards during the upcoming procedure.

Known calibration approaches however involve a manual intervention of medical personnel who bring the devices to be calibrated into alignment with the reference structures or calibration features. With the present invention it was found out that the technical capacity of medical technology and robotic systems can be better exploited in this regard so as to improve a calibration procedure, particularly in terms of comfort and efficiency.

The present invention has the object of improving and facilitating calibration of a medical support structure in a medical environment. The present invention can be used for any procedures that involve the use of motorized support structures including robotic arms. Aspects of the present invention, examples and exemplary steps and their embodiments are disclosed in the following. Different exemplary features of the invention can be combined in accordance with the invention wherever technically expedient and feasible.

### EXEMPLARY SHORT DESCRIPTION OF THE INVENTION

In the following, a short description of the specific features of the present invention is given which shall not be understood to limit the invention only to the features or a combination of the features described in this section.

The present invention provides an approach for calibrating a motorized medical support structure such as a medical robotic arm with respect to medical appliances and devices, wherein the support structure acts automatically so as to reach a calibration feature of a medical device it needs to be calibrated with.

### GENERAL DESCRIPTION OF THE INVENTION

In this section, a description of the general features of the present invention is given for example by referring to possible embodiments of the invention.

In general, the invention reaches the aforementioned object by providing, in a first aspect, a computer-implemented medical method of calibrating a motorized medical support structure. The method comprises executing, on at least one processor of at least one computer (for example at least one computer being part of a navigation system), the following exemplary steps which are executed by the at least one processor.

In a (for example first) exemplary step, presence data is acquired which describes the presence of the support structure and of the medical device within predefined spatial surroundings, particularly within the same treatment room or operating theatre.

In a (for example second) exemplary step, pre-positioning data is determined based on the presence data, which describes a spatial position of the medical device with respect to the support structure, in which the medical device is within a working range of the support structure.

In a (for example third) exemplary step, calibration target data is determined based on the pre-positioning data, which describes an expected spatial position of a calibration target of the medical device with respect to the support structure.

In a (for example fourth) exemplary step, reaching data is determined based on the calibration target data, which describes whether a calibration section of the support structure has reached the calibration target by being moved to the expected spatial position of the calibration target.

In a (for example fifth) exemplary step, guidance data is acquired in case the calibration section of the support structure has not reached the calibration target, which describes a necessary positional correction of the calibration section to reach the calibration target.

In a (for example sixth) exemplary step, calibration data is determined which describes a spatial relative position between the support structure and the medical device with the calibration section having reached the calibration target.

For calibrating the support structure which may be configured as articulated support structure and may further be referred to as “robotic arm” or “robot”, it is first determined whether there is a need for calibrating the support structure with other medical devices. Usually, this is the case when both of these systems are used for a planned medical procedure and, for example, are therefore present within the same treatment room or operating theatre. Thus, data, herein referred to as “presence data”, is acquired via one or more of the approaches described further below, wherein this data describes whether or not a calibration is needed.

In case it is determined that a calibration is needed, it is necessary for the following calibration to determine the relative position between the support structure and the device it needs to be calibrated with. As the support structure can perform the self-acting calibration only if it is able to reach the calibration target of the device, the device needs to be positioned within the support structure's working range first. If this is not the case, the device needs to be transferred into the working range prior to the calibration procedure. This can be done either manually by medical personnel which may move one or both of the device and the support structure, or automatically by for example a motorized undercarriage or trolley of either one of the device and the support structure.

The relative position between the device and the support structure may be tracked, for example via a conventional tracking system or one or more of the sensors described below, and it is therefore known from the received data when the device and the support structure have been positioned with respect to each other such that the device is within the working range of the support structure. The size and shape of the working range may for example be described by data acquired from a database. From the tracking data, the spatial relative position between the device and the support structure is also known, on which basis the support structure may perform its first attempt to reach the calibration target so as to align its calibration section with the calibration target. For doing so, the support structure is heading for the expected spatial position of the calibration target which may be derived from the data acquired so far. In case the calibration target or the calibration section are not tracked in a direct manner, but are variably or invariably coupled to tracked features, geometric properties of the device and/or of the support structure may also be taken into account, including pre-defined dimensions of individual parts of the device and/or of the support structure, and even the relative position of multiple sections thereof which can be variably coupled to each other.

In case the support structure is able to align its calibration section with the calibration target at the first try, for example with the calibration section resting at or in the calibration target of the device, the calibration has been brought to a successful end, such that for the upcoming procedure, the spatial position of the support structure along with its coordinate system can be determined with respect to the device with high accuracy. Any suitable sensor may be utilized to confirm whether the calibration section is successfully aligned with the calibration target.

However, if the first attempt of aligning the calibration section of the support structure with the calibration target is not successful, i.e. no confirmation is received from the one or more sensors, the calibration section needs to be transferred from its current position into the correct position of the calibration target. For doing so, guidance data is acquired on which basis the support structure is able to transfer its calibration section to the correct position of the calibration target. The guidance data is acquired via any conceivable sensor as described further below.

As soon as the calibration section has been transferred to the correct position and rests at or in the calibration target of the device, the calibration procedure has finally brought to a successful ending with the support structure being available for the following medical procedure.

It is however conceivable that the support structure may be calibrated with respect to any conceivable device or instrument to be used in a medical procedure along with the support structure.

In an example of the method according to the first aspect, the medical device is selected from the group consisting of:
a second, particularly articulated support structure, specifically a robotic support arm;
a medical imaging device, particularly wherein the support structure is calibrated with respect to an image space defined by the imaging device, and particularly with respect to at least one 2D- or 3D-image dataset acquired via the imaging device.

As was already indicated above, the presence data initially acquired may describe the spatial position of at least one of the support structure and the medical device, for example within a common coordinate system which may be assigned to the medical device or to the support structure. The presence data may further be acquired via a conventional tracking system known in the art, for example and optical, an electromagnetic or an ultrasound tracking system, or for example via at least one of:
an optical camera system assigned to the medical device and adapted to recognize the support structure;
an optical camera system assigned to the support structure and adapted to recognize the medical device;
an optical camera system assigned to a predefined spatial volume, particularly a treatment room or operating theatre, and adapted to recognize the medical device and/or the support structure;
a portable optical camera system assigned to a wearable device and adapted to recognize the medical device and/or the support structure;
a predefined treatment plan describing utilization of the medical device and/or the support structure;
a network based localization functionality describing the location of the medical device and/or the support structure within a hospital.

It is important to note here that the presence data does not necessarily need to describe the spatial position of the support structure or the medical device, but only needs to provide sufficient information to determine whether or not it is necessary or desired that the support structure is calibrated with a respective device. As already described further above, this is usually the case when the use of the respective device as well as of the support structure is planned for an upcoming procedure and/or when the respective device and the support structure are disposed within the same treatment room or operating theatre. Thus, it may be sufficient for the presence data to describe the “presence” of the respective device and the support structure in a confined space, for example the field of view of one or more optical sensors, without the necessity of also describing the spatial position of the support structure with respect to the respective device. As technically assisted medical procedures nowadays often involve the use of a large number of sensors and cameras for various purposes and tasks, the presence data may be acquired via any of these already available sensors or cameras without the need to provide additional equipment. It is also conceivable that the step of acquiring presence data involves a manual input describing the necessity of a calibration. Such input can be made by medical personnel via any device involved in the medical procedure, or connected thereto via a data link.

In a further example of the inventive method, determining pre-positioning data involves outputting control data for moving at least one of the support structure and the medical device with respect to each other to position the medical device at the spatial position within the working range of the support structure. On that basis, the support structure and the medical device are “coarsely” pre-positioned with respect to each other, such that the device is within the robotic arm's working range such that its calibration target can be reached by the support structure, particularly the calibration section thereof. Once the medical device has been pre-positioned within the working range of the support structure, which may be confirmed with the help of a tracking system or any other suitable sensor or even a manual input of medical personnel, the procedure may continue with moving the calibration section towards the calibration target.

In the same manner, determining calibration target data may involve outputting control data for moving the calibration section of the support structure to the expected spatial position of a calibration target of the medical device. This is considered the “fine”-positioning of the support structure along with its calibration section with respect to the medical device and its calibration target.

Any of the above control data may be output to at least one motor control unit for automatically moving at least one of the medical device and the support structure with respect to each other
to position the medical device at the spatial position within the working range of the support structure; and/or
to position the calibration section of the support structure in the expected spatial position of the calibration target.

While this is considered a fully automatic calibration procedure which may be performed without any manual interaction of medical personnel, the control data output in connection with the pre-positioning data may be also output to a user interface for instructing medical personnel to move at least one of the medical device and the support structure with respect to each other to position the medical device at the spatial position within the working range of the support structure, particularly wherein at least one of the medical device and the support structure is moved manually or via at least one manually controlled motor. This second alternative is considered a semi-automatic calibration procedure which for example may be performed in case the medical device and/or the support structure do not feature an automatically controlled and motorized undercarriage, so that the support structure and the medical device need to be manually pre-positioned with respect to each other. The user interface may for example include a graphical display which indicates how the support structure and the medical device need to be moved with respect to each other so as to dispose the medical device within the working range of the support structure.

In a further example of the inventive method, reaching data and/or guidance data may be acquired via at least one of:
at least one force sensitive sensor assigned to the support structure and adapted to sense a force acting on the support structure via the calibration section;
at least one force sensitive sensor assigned to the medical device and adapted to sense a force acting on the medical device via the calibration target and/or via a surface section of the medical device surrounding the calibration target;
an optical camera system, particularly a camera system of a medical tracking system and/or an optical camera system as described herein, adapted to recognize the calibration section and the calibration target;
at least one light sensitive sensor assigned to one of the medical device and the support structure and adapted to sense a laser-beam emitted via a laser-emitter assigned to the other one of the medical device and the support structure;
at least one ultrasound sensor, particularly disposed at the distal section of the support structure, and/or capable of determining the topography of the medical device and of the calibration target when being positioned in the vicinity thereof.

As was already described further above, any conceivable sensor may be utilized to provide the reaching data and/or the guidance data needed for the inventive method to be performed. As medical robots which may serve as a support structure within the framework of the present invention are regularly equipped with a vast number of sensors which are not only capable of observing the surroundings of the robot, but also of determining physical effects acting on the robot, the inventive approach may make use of those already existing sensors without the need of providing additional equipment for acquiring reaching data and/or guidance data.

In a further example of the inventive method, acquiring guidance data involves scanning, via the at least one sensor or the at least one camera system described above, a surface section of the medical device surrounding the calibration target and providing information as to the spatial position of the calibration target.

With one or more of the sensors described above, the support structure is capable of observing its immediate surroundings including the vicinity of the calibration target, which allows the support structure to find its way to the calibration target so as to place its calibration section there.

In a second aspect, the invention is directed to a computer program comprising instructions which, when the program is executed by at least one computer, causes the at least one computer to carry out the method according to the first aspect. The invention may alternatively or additionally relate to a (physical, for example electrical, for example technically generated) signal wave, for example a digital signal wave, such as an electromagnetic carrier wave carrying information which represents the program, for example the aforementioned program, which for example comprises code means which are adapted to perform any or all of the steps of the method according to the first aspect. The signal wave is in one example a data carrier signal carrying the aforementioned computer program. A computer program stored on a disc is a datafile, and when the file is read out and transmitted it becomes a data stream for example in the form of a (physical, for example electrical, for example technically generated) signal. The signal can be implemented as the signal wave, for example as the electromagnetic carrier wave which is described herein. For example, the signal, for example the signal wave is constituted to be transmitted via a computer network, for example LAN, WLAN, WAN, mobile network, for example the internet. For example, the signal, for example the signal wave, is constituted to be transmitted by optic or acoustic data transmission. The invention according to the second aspect therefore may alternatively or additionally relate to a data stream representative of the aforementioned program, i.e. comprising the program.

In a third aspect, the invention is directed to a computer-readable storage medium on which the program according to the second aspect is stored. The program storage medium is for example non-transitory.

In a fourth aspect, the invention is directed to at least one computer (for example, a computer), comprising at least one processor (for example, a processor), wherein the program according to the second aspect is executed by the processor, or wherein the at least one computer comprises the computer-readable storage medium according to the third aspect.

In a fifth aspect, the invention is directed to a medical system, comprising:
a) the at least one computer according to the fourth aspect;
b) the motorized medical support structure, particularly a medical robotic arm, according to the first aspect;
c) the medical device, particularly a medical imaging device, according to the first aspect;
wherein the computer is operably coupled to at least the support structure, particularly also to the medical device, for outputting control data to cause
   at least one of the support structure and the medical device to move with respect to each other to position the medical device at a spatial position within the working range of the support structure and/or
the calibration section of the support structure to move to the expected spatial position of a calibration target of the medical device.

In a further example of the inventive system, the support structure is transportable, particularly portable, specifically as an entire unit, and/or wherein the medical device is transportable, particularly as an entire unit, specifically via a wheeled undercarriage. In other words, the support structure may be configured to be carried by a human being and being clamped or otherwise mounted to a patient couch or other medical appliances in a desired manner. In the alternative, the support structure may be mounted on a wheeled trolley configured to move on the floor of a hospital. In the same manner, the medical device, particularly the medical imaging device may comprise a wheeled undercarriage to be moved on the floor of a hospital.

According to a further example, the support structure comprises a calibration section and the medical device comprises a corresponding calibration target, or vice versa, wherein the calibration target is adapted to receive the calibration section, particularly at a predefined position. For example, the calibration section may comprise a protrusion which fits exactly into a calibration target formed as a recess. In particular, the calibration target may receive the calibration section with no play, such that a precise relative position is established once the calibration section is received in the calibration target.

In a further example, a surface section of the medical device surrounding the calibration target is adapted to provide information as to the spatial position of the calibration target, particularly wherein the surface section includes at least one detectable marker indicating the spatial position of the calibration target with respect to the marker, specifically wherein at least one marker is
an optically detectable marker; and/or
a haptically detectable marker.

For example, the spatial relative position of the calibration target with respect to the marker may be codified in the marker's geometry. In case the calibration target is disposed at the center of a ring-shaped marker, or a plurality of concentric ring-shaped markers, the target's position can be easily calculated from the marker's curvature which may be detected by a sensor, even if the target itself is not recognized by the sensor. In further examples, the one or more markers may form a target cross with the target being disposed at the center. Additionally or alternatively to a ring shaped marker, one or more markers may form a grid, one or more point-shaped markings such as pins or dimples, or a plurality of markers which radially extend towards the target.

Alternatively or additionally, the invention according to the fifth aspect is directed to a for example non-transitory computer-readable program storage medium storing a program for causing the computer according to the fourth aspect to execute the data processing steps of the method according to the first aspect.

For example, the invention does not involve or in particular comprise or encompass an invasive step which would represent a substantial physical interference with the body requiring professional medical expertise to be carried out and entailing a substantial health risk even when carried out with the required professional care and expertise.

### Definitions

In this section, definitions for specific terminology used in this disclosure are offered which also form part of the present disclosure.

The method in accordance with the invention is for example a computer-implemented method. For example, all the steps or merely some of the steps (i.e. less than the total number of steps) of the method in accordance with the invention can be executed by a computer (for example, at least one computer). An embodiment of the computer implemented method is a use of the computer for performing a data processing method. An embodiment of the computer implemented method is a method concerning the operation of the computer such that the computer is operated to perform one, more or all steps of the method.

The computer for example comprises at least one processor and for example at least one memory in order to (technically) process the data, for example electronically and/or optically. The processor being for example made of a substance or composition which is a semiconductor, for example at least partly n- and/or p-doped semiconductor, for example at least one of II-, III-, IV-, V-, VI-semiconductor material, for example (doped) silicon and/or gallium arsenide. The calculating or determining steps described are for example performed by a computer. Determining steps or calculating steps are for example steps of determining data within the framework of the technical method, for example within the framework of a program. A computer is for example any kind of data processing device, for example electronic data processing device. A computer can be a device which is generally thought of as such, for example desktop PCs, notebooks, netbooks, etc., but can also be any programmable apparatus, such as for example a mobile phone or an embedded processor. A computer can for example comprise a system (network) of “sub-computers”, wherein each sub-computer represents a computer in its own right. The term “computer” includes a cloud computer, for example a cloud server. The term computer includes a server resource. The term “cloud computer” includes a cloud computer system which for example comprises a system of at least one cloud computer and for example a plurality of operatively interconnected cloud computers such as a server farm. Such a cloud computer is preferably connected to a wide area network such as the world wide web (WWW) and located in a so-called cloud of computers which are all connected to the world wide web. Such an infrastructure is used for “cloud computing”, which describes computation, software, data access and storage services which do not require the end user to know the physical location and/or configuration of the computer delivering a specific service. For example, the term “cloud” is used in this respect as a metaphor for the Internet (world wide web). For example, the cloud provides computing infrastructure as a service (IaaS). The cloud computer can function as a virtual host for an operating system and/or data processing application which is used to execute the method of the invention. The cloud computer is for example an elastic compute cloud (EC2) as provided by Amazon Web Services™. A computer for example comprises interfaces in order to receive or output data and/or perform an analogue-to-digital conversion. The data are for example data which represent physical properties and/or which are generated from technical signals. The technical signals are for example generated by means of (technical) detection devices (such as for example devices for detecting marker devices) and/or (technical) analytical devices (such as for example devices for performing (medical) imaging methods), wherein the technical signals are for example electrical or optical signals. The technical signals for example represent the data received or outputted by the computer. The computer is preferably operatively coupled to a display device which allows information outputted by the computer to be displayed, for example to a user. One example of a display device is a virtual reality device or an augmented reality device (also referred to as virtual reality glasses or augmented reality glasses) which can be used as “goggles” for navigating. A specific example of such augmented reality glasses is Google Glass (a trademark of Google, Inc.). An augmented reality device or a virtual reality device can be used both to input information into the computer by user interaction and to display information outputted by the computer. Another example of a display device would be a standard computer monitor comprising for example a liquid crystal display operatively coupled to the computer for receiving display control data from the computer for generating signals used to display image information content on the display device. A specific embodiment of such a computer monitor is a digital lightbox. An example of such a digital lightbox is Buzz®, a product of Brainlab AG. The monitor may also be the monitor of a portable, for example handheld, device such as a smart phone or personal digital assistant or digital media player.

The invention also relates to a computer program comprising instructions which, when on the program is executed by a computer, cause the computer to carry out the method or methods, for example, the steps of the method or methods, described herein and/or to a computer-readable storage medium (for example, a non-transitory computer-readable storage medium) on which the program is stored and/or to a computer comprising said program storage medium and/or to a (physical, for example electrical, for example technically generated) signal wave, for example a digital signal wave, such as an electromagnetic carrier wave carrying information which represents the program, for example the aforementioned program, which for example comprises code means which are adapted to perform any or all of the method steps described herein. The signal wave is in one example a data carrier signal carrying the aforementioned computer program. The invention also relates to a computer comprising at least one processor and/or the aforementioned computer-readable storage medium and for example a memory, wherein the program is executed by the processor.

Within the framework of the invention, computer program elements can be embodied by hardware and/or software (this includes firmware, resident software, micro-code, etc.). Within the framework of the invention, computer program elements can take the form of a computer program product which can be embodied by a computer-usable, for example computer-readable data storage medium comprising computer-usable, for example computer-readable program instructions, “code” or a “computer program” embodied in said data storage medium for use on or in connection with the instruction-executing system. Such a system can be a computer; a computer can be a data processing device comprising means for executing the computer program elements and/or the program in accordance with the invention, for example a data processing device comprising a digital processor (central processing unit or CPU) which executes the computer program elements, and optionally a volatile memory (for example a random access memory or RAM) for storing data used for and/or produced by executing the computer program elements. Within the framework of the present invention, a computer-usable, for example computer-readable data storage medium can be any data storage medium which can include, store, communicate, propagate or transport the program for use on or in connection with the instruction-executing system, apparatus or device. The computer-usable, for example computer-readable data storage medium can for example be, but is not limited to, an electronic, magnetic, optical, electromagnetic, infrared or semiconductor system, apparatus or device or a medium of propagation such as for example the Internet. The computer-usable or computer-readable data storage medium could even for example be paper or another suitable medium onto which the program is printed, since the program could be electronically captured, for example by optically scanning the paper or other suitable medium, and then compiled, interpreted or otherwise processed in a suitable manner. The data storage medium is preferably a non-volatile data storage medium. The computer program product and any software and/or hardware described here form the various means for performing the functions of the invention in the example embodiments. The computer and/or data processing device can for example include a guidance information device which includes means for outputting guidance information. The guidance information can be outputted, for example to a user, visually by a visual indicating means (for example, a monitor and/or a lamp) and/or acoustically by an acoustic indicating means (for example, a loudspeaker and/or a digital speech output device) and/or tactilely by a tactile indicating means (for example, a vibrating element or a vibration element incorporated into an instrument). For the purpose of this document, a computer is a technical computer which for example comprises technical, for example tangible components, for example mechanical and/or electronic components. Any device mentioned as such in this document is a technical and for example tangible device.

The expression “acquiring data” for example encompasses (within the framework of a computer implemented method) the scenario in which the data are determined by the computer implemented method or program. Determining data for example encompasses measuring physical quantities and transforming the measured values into data, for example digital data, and/or computing (and e.g. outputting) the data by means of a computer and for example within the framework of the method in accordance with the invention. A step of “determining” as described herein for example comprises or consists of issuing a command to perform the determination described herein. For example, the step comprises or consists of issuing a command to cause a computer, for example a remote computer, for example a remote server, for example in the cloud, to perform the determination. Alternatively or additionally, a step of “determination” as described herein for example comprises or consists of receiving the data resulting from the determination described herein, for example receiving the resulting data from the remote computer, for example from that remote computer which has been caused to perform the determination. The meaning of “acquiring data” also for example encompasses the scenario in which the data are received or retrieved by (e.g. input to) the computer implemented method or program, for example from another program, a previous method step or a data storage medium, for example for further processing by the computer implemented method or program. Generation of the data to be acquired may but need not be part of the method in accordance with the invention. The expression “acquiring data” can therefore also for example mean waiting to receive data and/or receiving the data. The received data can for example be inputted via an interface. The expression “acquiring data” can also mean that the computer implemented method or program performs steps in order to (actively) receive or retrieve the data from a data source, for instance a data storage medium (such as for example a ROM, RAM, database, hard drive, etc.), or via the interface (for instance, from another computer or a network). The data acquired by the disclosed method or device, respectively, may be acquired from a database located in a data storage device which is operably to a computer for data transfer between the database and the computer, for example from the database to the computer. The computer acquires the data for use as an input for steps of determining data. The determined data can be output again to the same or another database to be stored for later use. The database or database used for implementing the disclosed method can be located on network data storage device or a network server (for example, a cloud data storage device or a cloud server) or a local data storage device (such as a mass storage device operably connected to at least one computer executing the disclosed method). The data can be made “ready for use” by performing an additional step before the acquiring step. In accordance with this additional step, the data are generated in order to be acquired. The data are for example detected or captured (for example by an analytical device). Alternatively or additionally, the data are inputted in accordance with the additional step, for instance via interfaces. The data generated can for example be inputted (for instance into the computer). In accordance with the additional step (which precedes the acquiring step), the data can also be provided by performing the additional step of storing the data in a data storage medium (such as for example a ROM, RAM, CD and/or hard drive), such that they are ready for use within the framework of the method or program in accordance with the invention. The step of “acquiring data” can therefore also involve commanding a device to obtain and/or provide the data to be acquired. In particular, the acquiring step does not involve an invasive step which would represent a substantial physical interference with the body, requiring professional medical expertise to be carried out and entailing a substantial health risk even when carried out with the required professional care and expertise. In particular, the step of acquiring data, for example determining data, does not involve a surgical step and in particular does not involve a step of treating a human or animal body using surgery or therapy. In order to distinguish the different data used by the present method, the data are denoted (i.e. referred to) as “XY data” and the like and are defined in terms of the information which they describe, which is then preferably referred to as “XY information” and the like.

The n-dimensional image of a body is registered when the spatial location of each point of an actual object within a space, for example a body part in an operating theatre, is assigned an image data point of an image (CT, MR, etc.) stored in a navigation system.

Image registration is the process of transforming different sets of data into one co-ordinate system. The data can be multiple photographs and/or data from different sensors, different times or different viewpoints. It is used in computer vision, medical imaging and in compiling and analysing images and data from satellites. Registration is necessary in order to be able to compare or integrate the data obtained from these different measurements.

It is the function of a marker to be detected by a marker detection device (for example, a camera or an ultrasound receiver or analytical devices such as CT or MRI devices) in such a way that its spatial position (i.e. its spatial location and/or alignment) can be ascertained. The detection device is for example part of a navigation system. The markers can be active markers. An active marker can for example emit electromagnetic radiation and/or waves which can be in the infrared, visible and/or ultraviolet spectral range. A marker can also however be passive, i.e. can for example reflect electromagnetic radiation in the infrared, visible and/or ultraviolet spectral range or can block x-ray radiation. To this end, the marker can be provided with a surface which has corresponding reflective properties or can be made of metal in order to block the x-ray radiation. It is also possible for a marker to reflect and/or emit electromagnetic radiation and/or waves in the radio frequency range or at ultrasound wavelengths. A marker preferably has a spherical and/or spheroid shape and can therefore be referred to as a marker sphere; markers can however also exhibit a cornered, for example cubic, shape.

A marker device can for example be a reference star or a pointer or a single marker or a plurality of (individual) markers which are then preferably in a predetermined spatial relationship. A marker device comprises one, two, three or more markers, wherein two or more such markers are in a predetermined spatial relationship. This predetermined spatial relationship is for example known to a navigation system and is for example stored in a computer of the navigation system.

In another embodiment, a marker device comprises an optical pattern, for example on a two-dimensional surface. The optical pattern might comprise a plurality of geometric shapes like circles, rectangles and/or triangles. The optical pattern can be identified in an image captured by a camera, and the position of the marker device relative to the camera can be determined from the size of the pattern in the image, the orientation of the pattern in the image and the distortion of the pattern in the image. This allows determining the relative position in up to three rotational dimensions and up to three translational dimensions from a single two-dimensional image.

The position of a marker device can be ascertained, for example by a medical navigation system. If the marker device is attached to an object, such as a bone or a medical instrument, the position of the object can be determined from the position of the marker device and the relative position between the marker device and the object. Determining this relative position is also referred to as registering the marker device and the object. The marker device or the object can be tracked, which means that the position of the marker device or the object is ascertained twice or more over time.

The present invention is also directed to a navigation system for computer-assisted surgery. This navigation system preferably comprises the aforementioned computer for processing the data provided in accordance with the computer implemented method as described in any one of the embodiments described herein. The navigation system preferably comprises a detection device for detecting the position of detection points which represent the main points and auxiliary points, in order to generate detection signals and to supply the generated detection signals to the computer, such that the computer can determine the absolute main point data and absolute auxiliary point data on the basis of the detection signals received. A detection point is for example a point on the surface of the anatomical structure which is detected, for example by a pointer. In this way, the absolute point data can be provided to the computer. The navigation system also preferably comprises a user interface for receiving the calculation results from the computer (for example, the position of the main plane, the position of the auxiliary plane and/or the position of the standard plane). The user interface provides the received data to the user as information. Examples of a user interface include a display device such as a monitor, or a loudspeaker. The user interface can use any kind of indication signal (for example a visual signal, an audio signal and/or a vibration signal). One example of a display device is an augmented reality device (also referred to as augmented reality glasses) which can be used as so-called “goggles” for navigating. A specific example of such augmented reality glasses is Google Glass (a trademark of Google, Inc.). An augmented reality device can be used both to input information into the computer of the navigation system by user interaction and to display Information outputted by the computer.

The invention also relates to a navigation system for computer-assisted surgery, comprising:
a computer for processing the absolute point data and the relative point data;
a detection device for detecting the position of the main and auxiliary points in order to generate the absolute point data and to supply the absolute point data to the computer;
a data interface for receiving the relative point data and for supplying the relative point data to the computer and
a user interface for receiving data from the computer in order to provide information to the user, wherein the received data are generated by the computer on the basis of the results of the processing performed by the computer.

A navigation system, such as a surgical navigation system, is understood to mean a system which can comprise: at least one marker device; a transmitter which emits electromagnetic waves and/or radiation and/or ultrasound waves; a receiver which receives electromagnetic waves and/or radiation and/or ultrasound waves; and an electronic data processing device which is connected to the receiver and/or the transmitter, wherein the data processing device (for example, a computer) for example comprises a processor (CPU) and a working memory and advantageously an indicating device for issuing an indication signal (for example, a visual indicating device such as a monitor and/or an audio indicating device such as a loudspeaker and/or a tactile indicating device such as a vibrator) and a permanent data memory, wherein the data processing device processes navigation data forwarded to it by the receiver and can advantageously output guidance information to a user via the indicating device. The navigation data can be stored in the permanent data memory and for example compared with data stored in said memory beforehand.

In the field of medicine, imaging methods (also called imaging modalities and/or medical imaging modalities) are used to generate image data (for example, two-dimensional or three-dimensional image data) of anatomical structures (such as soft tissues, bones, organs, etc.) of the human body. The term “medical imaging methods” is understood to mean (advantageously apparatus-based) imaging methods (for example so-called medical imaging modalities and/or radiological imaging methods) such as for instance computed tomography (CT) and cone beam computed tomography (CBCT, such as volumetric CBCT), x-ray tomography, magnetic resonance tomography (MRT or MRI), conventional x-ray, sonography and/or ultrasound examinations, and positron emission tomography. For example, the medical imaging methods are performed by the analytical devices. Examples for medical imaging modalities applied by medical imaging methods are: X-ray radiography, magnetic resonance imaging, medical ultrasonography or ultrasound, endoscopy, elastography, tactile imaging, thermography, medical photography and nuclear medicine functional imaging techniques as positron emission tomography (PET) and Single-photon emission computed tomography (SPECT), as mentioned by Wikipedia.

The image data thus generated is also termed “medical imaging data”. Analytical devices for example are used to generate the image data in apparatus-based imaging methods. The imaging methods are for example used for medical diagnostics, to analyse the anatomical body in order to generate images which are described by the image data. The imaging methods are also for example used to detect pathological changes in the human body. However, some of the changes in the anatomical structure, such as the pathological changes in the structures (tissue), may not be detectable and for example may not be visible in the images generated by the imaging methods. A tumour represents an example of a change in an anatomical structure. If the tumour grows, it may then be said to represent an expanded anatomical structure. This expanded anatomical structure may not be detectable; for example, only a part of the expanded anatomical structure may be detectable. Primary/high-grade brain tumours are for example usually visible on MRI scans when contrast agents are used to infiltrate the tumour. MRI scans represent an example of an imaging method. In the case of MRI scans of such brain tumours, the signal enhancement in the MRI images (due to the contrast agents infiltrating the tumour) is considered to represent the solid tumour mass. Thus, the tumour is detectable and for example discernible in the image generated by the imaging method. In addition to these tumours, referred to as “enhancing” tumours, it is thought that approximately 10% of brain tumours are not discernible on a scan and are for example not visible to a user looking at the images generated by the imaging method.

Mapping describes a transformation (for example, linear transformation) of an element (for example, a pixel or voxel), for example the position of an element, of a first data set in a first coordinate system to an element (for example, a pixel or voxel), for example the position of an element, of a second data set in a second coordinate system (which may have a basis which is different from the basis of the first coordinate system). In one embodiment, the mapping is determined by comparing (for example, matching) the color values (for example grey values) of the respective elements by means of an elastic or rigid fusion algorithm. The mapping is embodied for example by a transformation matrix (such as a matrix defining an affine transformation).

### BRIEF DESCRIPTION OF THE DRAWINGS

In the following, the invention is described with reference to the appended figures which give background explanations and represent specific embodiments of the invention. The scope of the invention is however not limited to the specific features disclosed in the context of the figures, wherein

FIG. 1 shows an exemplary medical setup including an articulated robotic arm serving as support structure;

FIG. 2 shows a detailed view on the distal section of the robotic arm shown in FIG. 1;

FIG. 3 shows a first exemplary detailed view of the imaging device shown in FIG. 1;

FIG. 4 shows a second exemplary detailed view of the imaging device shown in FIG. 1; and

FIG. 5 illustrates the basic steps of the method according to the first aspect of the present invention.

### DESCRIPTION OF EMBODIMENTS

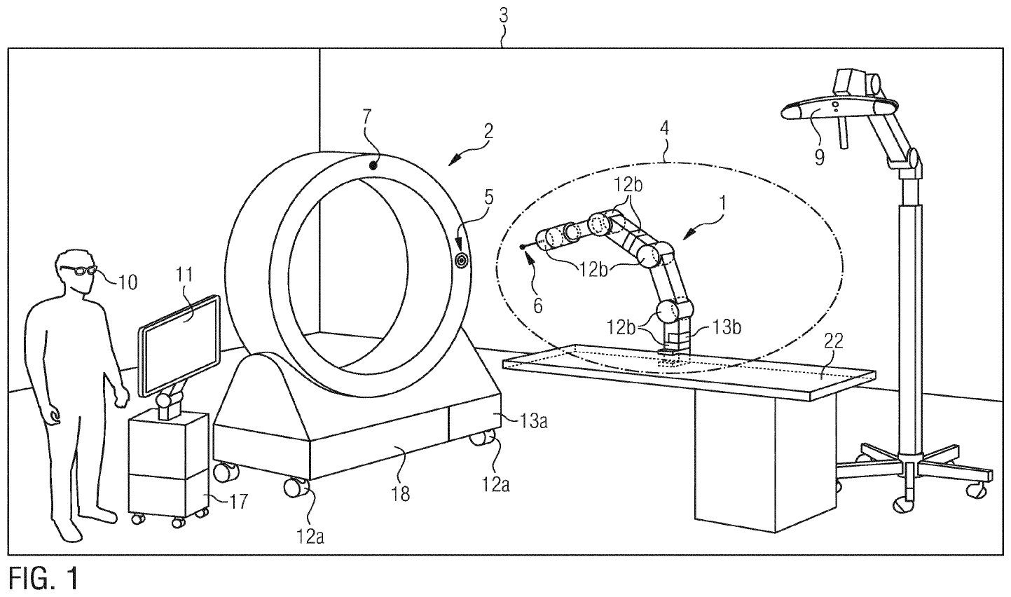

FIG. 1 shows an exemplary setup for a medical imaging procedure performed within a room 3 of a hospital. A CT-imaging device 2 is provided for acquiring a three-dimensional image dataset of a patient lying on a patient couch 22. For bringing the imaging device 2 into alignment with the patient, the imaging device 2 is provided with an undercarriage 18 having a plurality of wheels driven by motors 12a which are controlled via a motor control unit 13a.

On the other hand, the medical procedure to be performed on the patient lying on the patient couch 22 involves the use of a medical robotic arm that forms an articulated support structure 1 with a plurality of sections being connected via rotary joints which are driven by motors 12b controlled via a motor control unit 13b. While the support structure's proximal section is rigidly connected to the patient couch 22, its distal section features a calibration section 6 shaped as a longitudinal pointer with a distal tip that needs to be brought into alignment with a calibration target 5 of the device 2 for the support structure 1 to be calibrated with respect to the imaging device 2.

FIG. 1 schematically shows the working range 4 of the support structure 1, i.e. the spatial volume within which the support structure 1 is capable of placing the calibration section 6.

With the medical equipment provided within treatment room 3, a large variety of sensors is available for observing whether or not a calibration of the support structure 1 with respect to imaging device 2 is possible and desired. For example, an optical camera system 9 is provided as part of a medical tracking system. Medical personnel may wear AR-glasses that include a portable optical camera system 10. Either one of the support structure and the imaging device may include an optical camera system 8 and 7, respectively. Each one of those optical cameras provide images that can be searched via image registration for the support structure 1 and the imaging device 2 such that it can be determined therefrom whether or not the support structure 1 and the imaging device 2 are present within the same room 3 and need to be calibrated with each other. Further, an existing treatment plan may also indicate that both of the support structure 1 and the imaging device 2 are used for the same procedure and therefore need to be calibrated with each other.

In a second step, the calibration target 5 of imaging device 2 needs to be positioned within the working range 4 of the support structure 1 for the actual calibration procedure to start. Based on images provided via the stereoscopic camera array 9 a navigation system including computer 17 calculates and observes the relative position between the support structure 1 and the imaging device 2. Based on that information, control unit 13a of the imaging device 2 may control motors 12a of undercarriage 18 to move imaging device 2 towards robot 1 such that the calibration target 5 comes to rest within the working range 4. The navigation system includes a display 11 that may also be capable of providing information as to how medical personnel needs to position the imaging device 2 with respect to the support structure 1.

In a further step, calibration section 6 is moved by the support structure 1 to the expected spatial position of calibration target 5 which has been calculated from the positional data acquired via the navigation system and camera array 9. For doing so, the rotary joints of the support structure 1 are driven by motors which are in turn controlled by control unit 13b of support structure 1.

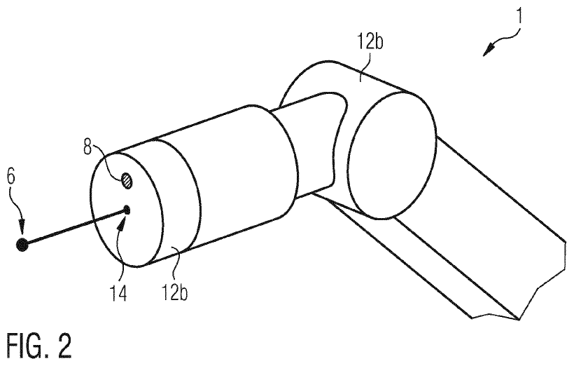

FIG. 2 shows the distal section of the support structure 1, which includes a force-sensitive sensor 14. Thus, any contact of the calibration section 6 with physical structures of the imaging device 2 will be recognized by sensor 14. Moreover, the distal section of the support structure 1 includes a camera 8 which observes the vicinity of the calibration section 6.

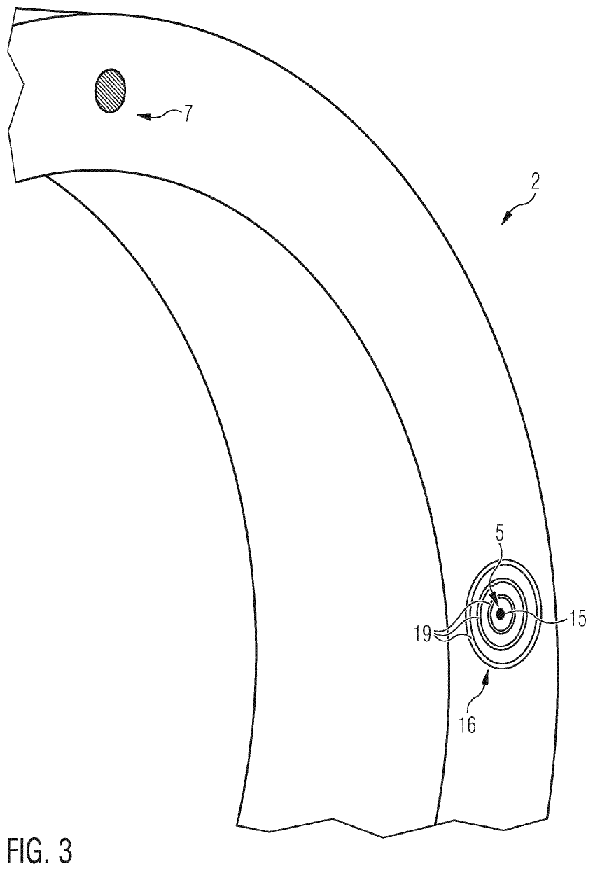

FIG. 3 shows that calibration target 5 of imaging device 2 is surrounded by a plurality of concentric ring-shaped protrusions or indentations which serve as optically and haptically detectable markers 19. Thus, in case the support structure 1 is not able to align the tip of calibration section 6 with the calibration target 5 which would be confirmed by push-button 15, camera 8 provides images which may be searched via image recognition for the ring-shaped markers 19 that are centered around target 5 and therefore indicate the distance as well as the direction in which the calibration section 6 needs to be moved so as to reach calibration target 5. In the same manner, data as to the distance and direction between the position of the calibration target 5 and the current position of the calibration section 6 may be acquired via sensor 14 when the tip of calibration section 6 is swept across the surface section 16 of imaging device 2 with ring markers 19 being haptically detected. In this way, the support structure 1 may “feel” its way along ring markers 19 until calibration section 6 reaches calibration target 5.

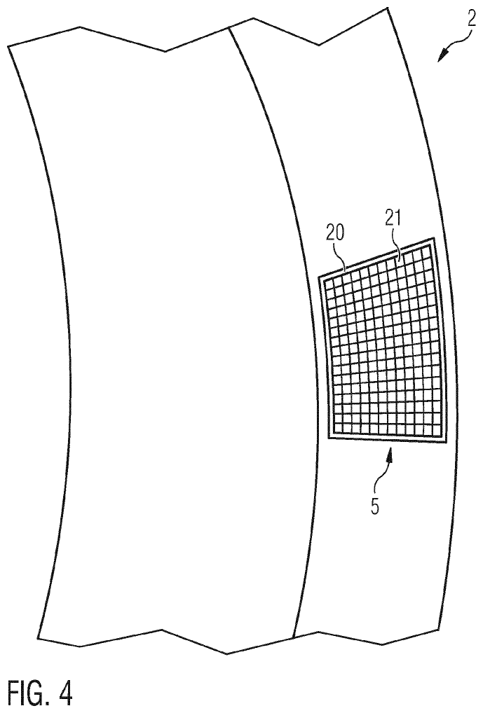

FIG. 4 shows another embodiment of imaging device 2 having a calibration target 5 that includes an array 20 of touch-sensitive sections 21, which is for example known from touch-sensitive displays. As soon as the tip of the calibration section 6 contacts the calibration target 5, irrespective of whether in the correct (expected) position or with some deviation therefrom, the calibration procedure is immediately completed without the need of guiding the tip of the calibration section 6 to the calibration target 5. Rather, the relative position between the tip of the calibration section 6 and the calibration target 5 is known from that first contact, such that transformations between the coordinate systems assigned to the imaging device 2 and the support structure 1, respectively, may be based on this determined relative position.

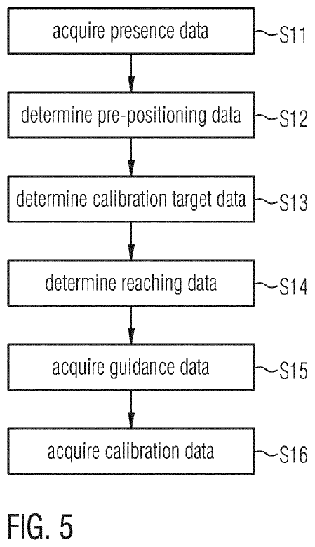

FIG. 5 illustrates the basic steps of the method according to the first aspect, in which step S11 encompasses acquiring presence data, step S12 encompasses determining pre-positioning data, step S13 encompasses determining calibration target data, Step S14 encompasses determining reaching data, step S15 encompasses acquiring guidance data and step S16 encompasses acquiring calibration data.

1. A computer implemented medical method of calibrating a motorized medical support structure with respect to a medical device, the method comprising:
acquiring presence data that describes a presence of the support structure and of the medical device within predefined spatial surroundings within a treatment room;
determining pre-positioning data based on the presence data, wherein the pre-positioning data describes a spatial position of the medical device with respect to the support structure in which the medical device is within a working range of the support structure;
determining calibration target data based on the pre-positioning data, wherein the calibration target data describes an expected spatial position of a calibration target of the medical device with respect to the support structure;
determining reaching data based on the calibration target data, wherein the reaching data describes whether a calibration section of the support structure has reached the calibration target by being moved to the expected spatial position of the calibration target;
acquiring guidance data based on the calibration section of the support structure not reaching the calibration target, wherein the guidance data describes a necessary positional correction of the calibration section to reach the calibration target; and
determining calibration data that describes a spatial relative position between the support structure and the medical device with the calibration section having reached the calibration target.

2. The method according to claim 1, wherein the medical device (2) is selected from the group consisting of:
a robotic support arm; and
a medical imaging device, wherein the support structure is calibrated with respect to an image space defined by the imaging device, and with respect to at least one 2D- or 3D-image dataset acquired via the imaging device.

3. The method according to claim 1, wherein the presence data describes the spatial position of at least one of the support structure and the medical device, and/or wherein the presence data is acquired via at least one of:
an optical camera system assigned to the medical device and adapted to recognize the support structure;
an optical camera system assigned to the support structure and adapted to recognize the medical device;
an optical camera system assigned to a predefined spatial volume comprising a treatment room or operating theatre, and adapted to recognize the medical device and/or the support structure;
a portable optical camera system assigned to a wearable device and adapted to recognize the medical device and/or the support structure;
a predefined treatment plan describing utilization of the medical device and/or the support structure; and/or
a network based localization functionality describing the location of the medical device and/or the support structure within a hospital.

4. The method according to claim 1, wherein the determining the pre-positioning data comprises outputting control data for moving at least one of the support structure and the medical device with respect to each other to position the medical device at the spatial position within the working range of the support structure.

5. The method according to claim 1, wherein the determining the calibration target data comprises outputting control data for moving the calibration section of the support structure to the expected spatial position of a calibration target of the medical device.

6. The method according to claim 4, wherein the control data is output via a user interface for instructing medical personnel to move at least one of the medical device and the support structure with respect to each other to position the medical device at the spatial position within the working range of the support structure, wherein at least one of the medical device and the support structure is moved manually or via at least one manually controlled motor.

7. The method according to claim 6, wherein the control data is output to at least one motor control unit for automatically moving at least one of the medical device and the support structure with respect to each other
to position the medical device at the spatial position within the working range of the support structure; and/or
to position the calibration section of the support structure in the expected spatial position of the calibration target.

8. The method according to claim 1, wherein the reaching data and/or the guidance data is acquired via at least one of:
at least one force sensitive sensor assigned to the support structure and adapted to sense a force acting on the support structure via the calibration section;
at least one force sensitive sensor assigned to the medical device and adapted to sense a force acting on the medical device via the calibration target and/or via a surface section of the medical device surrounding the calibration target;
an optical camera system comprising a camera system of a medical tracking system and/or an optical camera system adapted to recognize the calibration section and the calibration target; and/or
at least one light sensitive sensor assigned to one of the medical device and the support structure and adapted to sense a laser-beam emitted via a laser-emitter assigned to the other one of the medical device and the support structure.

9. The method according to claim 1, wherein the acquiring the guidance data comprises scanning, via the at least one sensor or the at least one camera system a surface section of the medical device surrounding the calibration target and providing information as to the spatial position of the calibration target.

10. A computer program comprising logic instructions stored on a non-transient storage medium that when the program is executed by a processor of a computer cause the computer to carry out a method of calibrating a motorized medical support structure with respect to a medical device, the method comprising:
acquiring presence data that describes a presence of the support structure and of the medical device within predefined spatial surroundings within a treatment room;
determining pre-positioning data based on the presence data, wherein the pre-positioning data describes a spatial position of the medical device with respect to the support structure in which the medical device is within a working range of the support structure;
determining calibration target data based on the pre-positioning data, wherein the calibration target data describes an expected spatial position of a calibration target of the medical device with respect to the support structure;
determining reaching data based on the calibration target data, wherein the reaching data describes whether a calibration section of the support structure has reached the calibration target by being moved to the expected spatial position of the calibration target;
acquiring guidance data based on the calibration section of the support structure not reaching the calibration target, wherein the guidance data describes a necessary positional correction of the calibration section to reach the calibration target; and
determining calibration data that describes a spatial relative position between the support structure and the medical device with the calibration section having reached the calibration target.

11. A medical system comprising:
a computer comprising a processor, a non-transient memory device, and logic instructions stored on the non-transient memory device;
a motorized medical support structure; and
a medical device comprising a medical imaging device;
wherein the computer is operably coupled to at least the support structure and the medical device, for outputting control data to cause:
at least one of the support structure and/or the medical device to move with respect to each other to position the medical device at a spatial position within a working range of the support structure; and/or
the calibration section of the support structure to move to the expected spatial position of a calibration target of the medical device.

12. The system according to claim 11, wherein the support structure (1) is transportable, particularly portable, specifically as an entire unit, and/or wherein the medical device (2) is transportable, particularly as an entire unit, specifically via a wheeled undercarriage (18).

13. The medical system according to claim 11, wherein the support structure comprises a calibration section and the medical device comprises a corresponding calibration target adapted to receive the calibration section at a predefined position.

14. The medical system according to claim 11, wherein a surface section of the medical device surrounding the calibration target is adapted to provide information as to the spatial position of the calibration target, wherein the surface section comprises at least one detectable marker indicating the spatial position of the calibration target with respect to the marker, wherein the at least one marker comprises:
an optically detectable marker; and/or
a haptically detectable marker.

15. The medical system according to claim 11, wherein the calibration target comprises an array of sensors adapted to detect the spatial position of a contact or an encounter with the calibration section of the support structure, wherein the plurality of sensors comprises:
at least one force sensitive sensor; and/or
at least one optically sensitive sensor.
